(19) United States
(12) Patent Application Publication
(10) Pub. No.: US 2009/0314287 A1
(43) Pub. Date: Dec. 24, 2009

(54) PORTABLE INHALER WITH ADD-ON DEVICE WITH A CHAMBER FOR INTERMEDIATE STORAGE OF AN ATOMIZED MEDICAMENT PREPARATION

(75) Inventors: Michael SPALLEK, Ingelheim am Rhein (DE); Herbert WACHTEL, Ingelheim am Rhein (DE); Deborah BICKMANN, Urbar (DE)

Correspondence Address: ROBERTS MLOTKOWSKI SAFRAN & COLE, P.C.;Intellectual Property Department, P.O. Box 10064, MCLEAN, VA, 22102-8064, US

(73) Assignee: BOEHRINGER INGELHEIM INTERNATIONAL GMBH, Ingelheim am Rhein (DE)

(21) Appl. No.: 12/487,840

(22) Filed: Jun. 19, 2009

(30) Foreign Application Priority Data
Jun. 20, 2008 (EP) 08011228.7

(51) Int. Cl. A61M 11/02

(52) U.S. Cl. 128/200.14

(57) ABSTRACT

An inhaler for propellant-free atomization of a medicament preparation. The inhaler produces an aerosol at low speed. The inhaler is combined with an add-on device for intermediate storage of the aerosol produced, so as to allow easier inhalation, particularly for children.

### BACKGROUND OF THE INVENTION

1. Field of Invention

The present invention relates to a portable inhaler for the propellant-free atomization of a medicament preparation, and in particular, to an add-on device or the combination of an add-on device with the inhaler, the add-on device having a chamber for the intermediate storage of the atomized medicament preparation.

2. Description of Related Art

The present invention relates, in particular, to a so-called soft mist inhaler (SMI), i.e., an inhaler of the type which produces only a relatively slowly spreading spray mist (aerosol). Inhalers of this kind, for the purposes of the present invention, are in particular inhalers in which an aerosol is delivered at a speed of less than 2 m/s, preferably less than 1.5 m/s and most preferably less than 1 m/s (in each case measured at a distance of 10 cm from a dispensing nozzle).

The starting point of the present invention is an inhaler as described in principle in International Patent Application Publication WO 91/14468 A1 and specifically as shown in FIGS. 6a & 6b of International Patent Application Publication WO 97/12687 A1 and corresponding U.S. Pat. No. 5,964,416. The known inhaler comprises as a reservoir for a medicament preparation which is to be atomized an insertable, rigid container having an inner bag containing the medicament preparation and a pressure producing means having a drive spring for conveying and atomizing the medicament preparation. Atomization is carried out without the use of a propellant gas, namely by the force of the drive spring. This inhaler is an SMI in the sense of the present invention.

A problem with inhalers and SMIs in general is that the triggering of the atomization of the medicament preparation and breathing in have to be coordinated. This may be difficult for the individual user. In particular, it has been found that such coordination is very difficult specifically for children. Studies have shown that the aerosol produced by unskilled users or, for example, children are often not optimally inhaled.

International Patent Application Publication WO 2004/091704 A1 discloses an add-on device for the intermediate storage of an atomized medicament preparation in a chamber. The add-on device is used in a so-called metered dose inhaler (MDI). The MDI comprises a pressurized container which contains the medicament preparation that is to be atomized and propellant gas. On actuation, the medicament preparation is expelled by means of the propellant gas at comparatively high pressure and correspondingly high speed and with a high mass flow. As a result, the expulsion is very brief, lasting, in particular, for less than 0.4 s, usually for about 0.15 to 0.36 s. The short expulsion time is disadvantageous for inhalation as the process of breathing in for inhalation usually lasts considerably longer. The comparatively high speed of more than 2 m/s often even above 8 m/s, at which the aerosol is usually delivered by an MDI, is also disadvantageous for receiving it into the lungs as the particles (droplets) of the aerosol are largely deposited on the walls of the user's throat as a result of the high speed during direct inhalation.

The known add-on device is provided for an MDI and serves to slow down the aerosol, particularly by lengthening the flow path. For this reason, add-on devices of this kind are also known as spacers. Moreover, the add-on device serves for intermediate storage of the aerosol produced so that the user has sufficient time to inhale the aerosol.

### SUMMARY OF THE INVENTION

The object of the present invention is to provide an inhaler, most preferably an SMI, and the use of an add-on device with a chamber for intermediate storage of an atomized medicament preparation, thereby making it possible to simplify inhalation even of aerosols delivered at low speed and/or preventing or at least minimizing problems occurring in the coordination of breathing in and the operation of an inhaler.

The present invention is based on the idea of combining a portable atomizer for propellant-free atomizing of a medicament preparation or an SMI with an add-on device comprising a chamber for intermediate storage of the aerosol produced, the chamber being arranged downstream of a delivery nozzle of the inhaler.

It has been found that thanks to the add-on device, even in an inhaler which produces the aerosol that is to be inhaled over a comparatively long time, preferably more than 1 second, and/or at comparatively low speed, preferably less than 2 m/s, most preferably less than 1.5 m/s (measured at a distance of 10 cm from a delivery nozzle), it is possible to achieve surprisingly improved inhalation of the active substance, particularly in small children or other people who have problems of coordination. Coordinating the actuation of the inhaler, i.e., the production of the aerosol, and breathing in is made substantially easier. The aerosol is produced by the inhaler and sprays into the chamber of the add-on device. The user can then inhale the aerosol by breathing in as deeply as possible, but without any compulsion of coordination or synchronization.

The proposed solution allows better defined inhalation of the active substance with a content of active substance which is, in the last analysis, higher on average and/or fluctuates less, this active substance being deposited in the lungs. This allows improved therapy of children and/or a broadening of the indication or the use of other medicament preparations. Thus, in the last analysis, it renders a propellant free inhaler or SMI universally usable.

Preferably, the add-on device has a valve so as to prevent the user from breathing out into the inhaler or chamber, i.e., to prevent air from flowing back from the delivery side of the add-on device into the chamber.

Further advantages, features, properties and aspects of the present invention will become apparent from the following description of a preferred embodiment with reference to the accompanying drawings.

### BRIEF DESCRIPTION OF THE DRAWINGS

FIG. 1 is a schematic section through an inhaler in the relaxed state;

FIG. 2 is a schematic section through the inhaler, rotated through 90° relative to FIG. 1, in the tensioned state;

FIG. 3 is a schematic section through the inhaler, corresponding to FIG. 1, with the add-on device attached;

FIG. 4 is a schematic exploded view of the atomizer and the add-on device with different accessories; and

FIG. 5 is a chart reflecting test results.

### DETAILED DESCRIPTION OF THE INVENTION

In the figures, the same reference numerals have been used for identical or similar parts where corresponding or comparable properties and advantages are achieved, even if the relevant description has not been repeated.

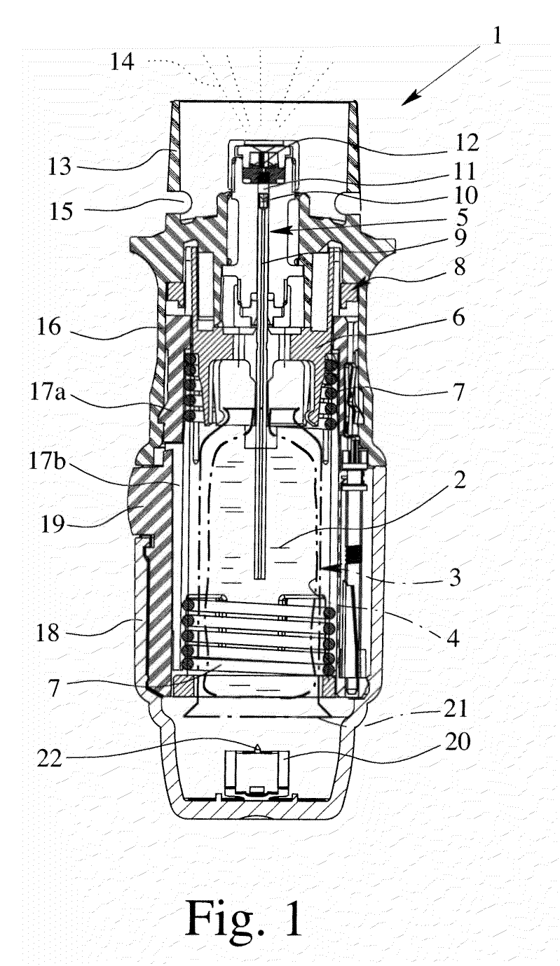

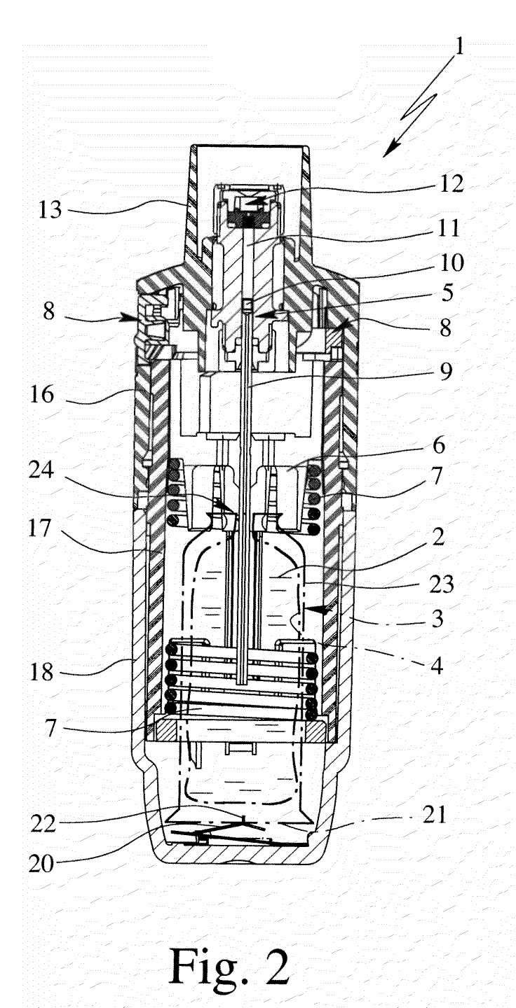

FIGS. 1 & 2 show a proposed portable inhaler 1 for the propellant free atomization of a medicament preparation 2 in a schematic view in the relaxed state (FIG. 1) and in the tensioned state (FIG. 2). FIGS. 1 & 2 show the inhaler 1 with a container 3 holding the medicament preparation 2.

During the atomization of the medicament preparation 2, preferably a liquid, a respirable aerosol 14 (FIG. 1) is formed which can be breathed in or inhaled by a user or patient (not shown). Normally, inhalation takes place at least once a day, but particularly several times a day, preferably at specified intervals of time, more particularly depending on the complaint suffered by the patient.

The inhaler 1 comprises the preferably insertable and optionally exchangeable container 3 holding the medicament preparation 2. The container 3 thus forms a reservoir for the medicament preparation 2 which is to be atomized. Preferably, the container 3 contains a sufficient quantity of medicament preparation 2 or active substance for several doses of the medicament preparation 2, i.e., to allow a number of atomizations or applications. A typical container 3 as disclosed in International Patent Application Publication WO 96/06011 A1 and corresponding U.S. Pat. No. 5,833,088 holds a volume of about 2 to 10 ml. With regard to the preferred construction of the container 3 reference is additionally made to International Patent Application Publication WO 00/49988 A2.

The container 3 is preferably substantially cylindrical or cartridge-shaped and can be inserted into the inhaler 1 from below, after it has been opened, and optionally exchanged. It is preferably of rigid construction, the medicament preparation 2 being contained in particular in a collapsible bag 4 in the container 3.

The inhaler 1 also comprises a conveying device, particularly a pressure generator 5, for conveying and atomizing the medicament preparation 2, particularly in a predetermined and optionally adjustable dosage amount in each case.

The inhaler 1 or pressure generator 5 comprises, in particular, a holder 6 for the container 3 and associated drive spring 7 which is only partly shown, preferably having an associated locking element 8 which is manually operable to release it, a conveying element, preferably a conveying tube 9 in the form of a capillary, with an optional valve, particularly a non-return valve 10, a pressure chamber 11 and/or a delivery nozzle 12, particularly in the region of a mouthpiece 13.

The container 3 is fixed in the inhaler 1 by means of the holder 6, particularly by a clamping or latching action, such that the conveying tube 9 protrudes into the container 3. The holder 6 may be constructed such that the container 3 can be exchanged.

When the drive spring 7 is axially tensioned, the holder 6 with the container 3 and the conveying tube 9 is moved downwards in the figures and the medicament preparation 2—or more precisely the next dose—is sucked out of the container 3 into the pressure chamber 11 of the pressure generator 5 through the non-return valve 10.

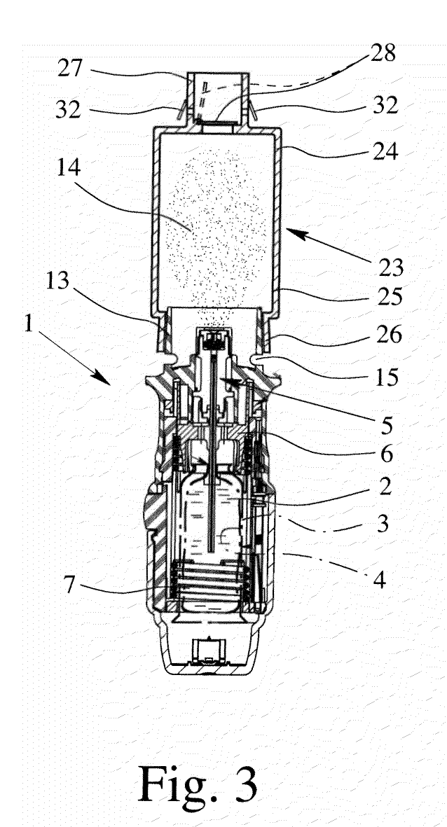

During the subsequent release of tension after actuation of the locking element 8, the medicament preparation 2 in the pressure chamber 11 is placed under pressure by moving the conveying tube 9 back up, with the non-return valve 10 now closed, by releasing the tension on the drive spring 7, so that this conveying tube 9 now acts as a pressure ram. This pressure expels the medicament preparation 2 through the delivery nozzle 12, where it is atomized into the preferably respiratable aerosol 14, as shown in FIGS. 1 and 3.

The user or patient (not shown) can inhale the aerosol 14, while preferably supply air can be sucked into the mouthpiece 13 through at least one supply air opening 15.

During the atomization process, the container 3 is moved back into its original position by the drive spring 7. Thus, the container 3 performs a lifting movement during the tensioning process and during the atomization process.

The inhaler 1 comprises, in particular, a first housing part (upper part) 16 and an inner part 17 which is rotatable relative thereto (FIG. 2) having an upper part 17a and a lower part 17b (FIG. 1), while a second housing part (lower part) 18, which is in particular manually operable or rotatable, is releasably attached, in particular pushed onto the inner part 17, preferably by means of a safety closure or retaining element 19. In particular, the safety closure or retaining element 19 is constructed such that accidental opening of the inhaler 1 or removal of the second housing part 18 is prevented. In particular, in order to release the second housing part 18, the retaining element 19 has to be pressed in against spring force. In order to insert and/or replace the container 3, the second housing part 18 can be detached from the inhaler 1. The second housing part 18 preferably forms a cap-like lower housing part and/or engages around or over a lower free end portion of the container 3.

The second housing part 18 can be rotated relative to the first housing part 16, whereby the inner part 17 is also rotated. In this way, the drive spring 7 is tensioned in the axial direction by means of a gear (not shown in detail) acting on the holder 6. During tensioning, the container 3 is moved axially downwards or with its end portion (further) into the second housing part 18 or towards the end face thereof, until the container 3 assumes the end position shown in FIG. 2. In this state, the drive spring 7 or inhaler 1 is clamped and locked.

The inhaler 1 preferably has a device for forcibly ventilating the container 3.

When tensioning first takes place, the container 3 is preferably pierced in its base or opened. In particular, an axially acting spring 20 arranged in the housing part 18 comes to abut on the container base 21 and with a piercing element 22 pierces the container 3 or an in particular gas tight seal provided in the base for ventilation purposes when contact is first made.

The device for forcible ventilation is thus formed in this case by the piercing element 22, which is held or formed by the spring 20. However, other design solutions are also possible.

It is noted that only the outer shell of the container 3 is opened during the piercing for ventilation purposes. The bag 4 containing the medicament preparation 2 remains undamaged. As the medicament formulation 2 is removed from the bag 4 through the conveying tube 9, the flexible bag 4 collapses. For pressure equalization, ambient air can flow into the container 3 through the ventilation or piercing opening.

In order to use the inhaler 1, first of all, the container 3 has to be inserted. This is preferably done by removing or pulling out the second housing part 18. The container 3 is then axially inserted or pushed into the inner part 17. At the same time, the container 3 is opened at the head end or attached. This is done by means of the conveying element, i.e., the conveying tube 9, which pierces a seal preferably provided at the head end of the container 3 and is then inserted through a septum at the head end of the container 3 into the interior of the bag 4. Thus, the fluidic connection between the container 3, or more accurately between the bag 4 in the container 3, is produced via the conveying tube 9 to the pressure generator 5 or pressure chamber 11.

Then, the second housing part 18 is pushed on again. The inhaler 1 can now be tensioned for the first time. At this stage, the container 3 is then pierced at its base by the piercing element 22, i.e., forcibly ventilated, as explained previously.

Before being used for the first time and after the container 3 has been inserted and fluidically connected, the inhaler 1 is preferably tensioned and actuated several times. This so-called priming displaces any air present in the medicament preparation 2 in the conveying tube 9 and in the pressure generator 5 to the delivery nozzle 12. The inhaler 1 is then ready for inhalation.

The quantity of medicament preparation 2 delivered per spray or atomization process is preferably about 10 μl to 50 μl, more particularly about 10 μl to 20 μl, most preferably about 15 μl.

The drive spring 7 is preferably installed in a biased state in order to achieve a high spring pressure. In the proposed inhaler 1, the pressurization and conveying of the medicament preparation 2 during the atomization process namely takes place preferably only by spring force, and more particularly only by the force of the drive spring 7.

The inhaler 1 is preferably constructed such that the medicament preparation 2 in the pressure generator 5 or in the pressure chamber 11 reaches a pressure of 5 MPa to 60 MPa, particularly about 10 MPa to 50 MPa during delivery. Particularly preferably, during the delivery or atomization of the medicament preparation 2, a pressure of about 5 MPa to 60 MPa, more particularly about 10 to 30 MPa, is reached at the delivery nozzle 12 or at the nozzle openings thereof. The medicament preparation 2 is then converted into the aerosol 14, the droplets of which have an aerodynamic diameter of up to 20 μm, preferably about 3 μm to 10 μm. The atomizing activity or atomizing effect is achieved or further assisted by preferably intercepting jets delivered by the delivery nozzle 12.

The inhaler 1 is preferably constructed such that the aerosol 14 is delivered at low speed, particularly at a speed of less than 2 m/s, most preferably about 1.6 m/s or less (in each case measured at a distance of 10 cm from the delivery nozzle 12). The inhaler 1 is thus preferably in the form of an SMI. The low delivery speed can be obtained or assisted by intercepting jets of the medicament preparation 2, which are delivered by the delivery nozzle 12 and/or by a suitable choice of spring force.

Particularly preferably, the construction of the inhaler 1 is such that the aerosol generation lasts for more than 0.7 s, more preferably at least about 1 s and in particular at least 1.5 s. The time taken to atomize a dose or to actuate the inhaler 1 is thus at least 1 s, more particularly more than 1.5 s.

The inhaler 1 has an add-on device 23 with a chamber 24 for intermediate storage of the aerosol 14 produced by the inhaler 1, as is shown in a schematic section in FIG. 3.

The chamber 24 is arranged or adapted to be arranged downstream of the delivery nozzle 12. It serves to receive and intermediately store the aerosol 14 produced by the inhaler 1.

Preferably, the add-on device 23 or its chamber 24 is at least substantially cylindrical, elongate or conical in construction.

Preferably, the chamber 24 is of larger cross section than the mouthpiece 13 of the inhaler 1 and/or it widens out at least in parts towards the delivery end or free end of the add-on device 23. This ensures that the aerosol 14 strikes a wall of the chamber 24 over the smallest possible area. In this way it is possible to minimize the deposition or settling of the atomized medicament preparation 2 on the wall of the chamber.

The chamber 24 preferably has a volume of more than 0.1 l, particularly more than 0.2 l, most preferably about 0.2 to 0.6 l. In particular, the add-on device 23 or the size of the chamber 24 is adapted to the inhaler 1 such that the aerosol 14 produced on actuation of the inhaler 1 can be at least substantially entirely received by the chamber 24 without the aerosol 14 or the atomized medicament preparation 2 essentially being deposited or settling on the inner wall of the chamber.

The add-on device 23 in the embodiment shown preferably comprises a housing 25 which is in particular elongate and/or cylindrical in construction.

The add-on device 23 or its housing 25 is preferably at least substantially rigid in construction. However, the add-on device 23, the chamber 24 or the housing 25 may theoretically also be flexible, inflatable and/or telescopic in construction, in order to minimize the space taken up when not in use and/or for transportation purposes, in particular.

The add-on device 23 or the housing 25 preferably has a connecting member 26 for connecting to the inhaler 1, particularly the mouthpiece 13 thereof.

Preferably, the add-on device 23 or the connecting member 26 can be fitted onto the mouthpiece 13, particularly by a clamping effect, and/or can be released again from the inhaler 1 or mouthpiece 13. However, the add-on device 23 may if necessary be connected or connectable to the inhaler 1 in a fixed or non-removable manner.

In the embodiment shown, the mouthpiece 13 preferably has at least one supply air opening 15. Particularly preferably, at least one supply air opening 15 remains open when the add-on device 23 has been attached, particularly fitted on, as shown in FIG. 3. This may be achieved for example by corresponding conical design of the mouthpiece 13 and a complementary design of the connecting member 26, by a stop (not shown) and/or other design features.

Preferably, the add-on device 23 is not rotatable relative to the inhaler 1. This is achieved in the embodiment by designing the mouthpiece 13 of the inhaler 1 with a noncircular, but preferably oval, outer contour to which the connecting member 26 is matched accordingly. However, other design solutions are also possible.

The add-on device 23 or housing 25 preferably comprises a delivery member 27 for delivering the aerosol 14. The delivery member 27 is preferably arranged on the end of the chamber 24 or of the housing 25 which is opposite the connecting member 26.

Particularly preferably, the connecting member 26 and delivery member 27 are formed in one piece with the housing 25. However, other design solutions are also possible.

Preferably, the chamber 24 or the housing 25 is at least partly or totally transparent in construction. This assists cleaning, in particular.

The add-on device 23 preferably has, at the delivery end, a valve 28 for preventing air from flowing back into the chamber 24. In this way, it is possible to prevent air from flowing into the chamber 24 as the user breathes out, which would force out the aerosol 14 through the attached mouthpiece 13 and the supply air openings 15, for example.

The valve 28 is preferably a non-return valve.

Particularly preferably, the valve 28 is incorporated in the connecting member 27. Particularly preferably, the valve 28 can be detached from the add-on device 23 or the housing 25, for example for cleaning purposes.

Alternatively or in addition to the valve 28, the proposed inhaler 1 may also have a valve device (not shown) in the region of the mouthpiece 13 and/or the supply air opening or openings 15 to prevent air from flowing back out of the chamber 24 through the mouthpiece 13 and out through the supply air opening or openings 15.

Alternatively or in addition, the add-on device 23 may also have an additional valve device (not shown), for example, between the connecting member 26 and the mouthpiece 13, to allow air to flow into the chamber 24, but prevent it from flowing out. In this case, the supply air openings 15 may be dispensed with altogether and/or may be covered or closed off by the add-on device 24.

Preferably, the add-on device 23 comprises, alternatively or in addition, at least one valve 32 for blowing out the exhaled air, as schematically shown in FIG. 3. In particular, FIG. 3 shows two such valves 32 in the opened state. Valve flaps are lifted away from the associated outlet openings. The at least one valve 32 is preferably at the delivery end, and in particular, is arranged on the delivery member 27. However, other design solutions are also possible.

When a user (not shown) breathes in, the valve 28 opens, as shown by broken lines in FIG. 3. The valves 32 are closed. The aerosol 14 is sucked out of the chamber 24 and emitted through the delivery member 27. As the user breathes out, the valve 28 closes or is closed. The valves 32 open and allow the exhaled air to be blown out without this air flowing into the chamber 24 or affecting the aerosol 14.

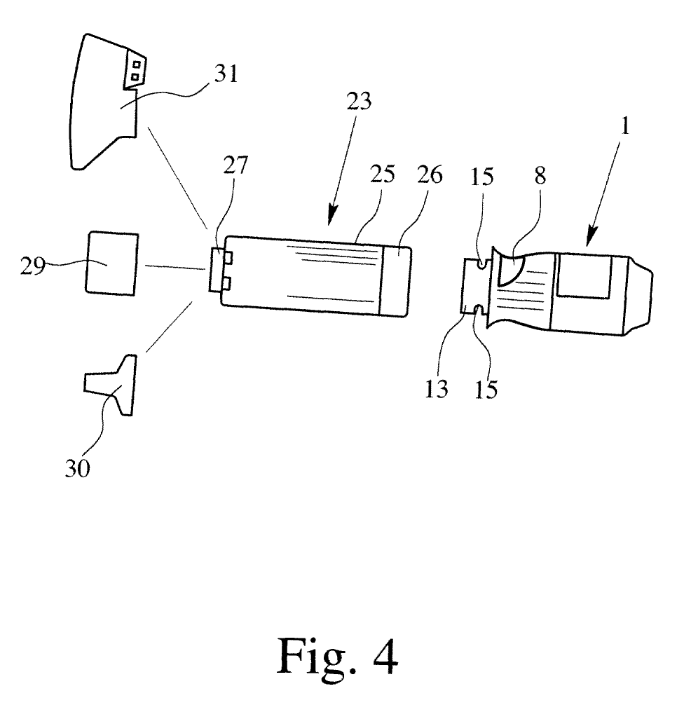

The add-on device 23 or its delivery member 27 may be equipped at the delivery end, preferably with an add-on mouthpiece 29, a tube 30 and/or a face mask 31, as shown by way of example in FIG. 4. In particular, different end pieces such as the add-on mouthpiece 29, the tube 30 and/or the face mask 31 can be selectively attached to the add-on device 23 or its delivery member 27, most preferably by fitting on.

Tests have shown that the proposed use of the add-on device 23 with the inhaler 1, i.e., the proposed intermediate storage of the aerosol 14 produced by the inhaler 1 in a sufficiently large chamber 24 may substantially contribute to a higher proportion of the active substance being received in the lungs on inhalation, even where there are problems of co-ordination.

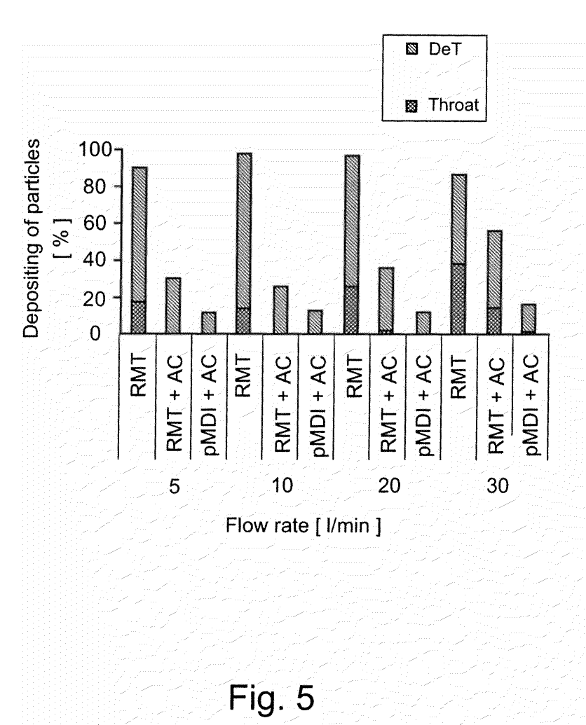

FIG. 5 illustrates the proportion of active substance which is supplied for the lungs as a whole (DeT) and the proportion of active substance deposited in the throat (Throat) as a function of the overall dosage amount for different inhalers.

The vertical axis shows the percentage amount of the respective dose which is actually delivered, while DeT shows the proportion which is made available to the lungs on inhalation, and Throat indicates the proportion which is deposited in the throat. The deposition of active substance was determined on the basis of the declared content using a Finlay throat model. An inhaled volume of 0.5 l in all was taken as the basis.

The tests were carried out for different apparatus or combinations of apparatus and at different flow rates, as plotted on the horizontal axis. RMT indicates the results of the inhaler 1 without the add-on device 23. RMT+AC indicates the results when the inhaler 1 is combined with the add-on device 23. pMDI+AC gives the results when a conventional MDI is combined with the add-on device 23. The numbers 5, 10, 20 and 30 each indicate the flow rate in l/min.

FIG. 5 shows that the use of the add-on device 23 leads to a reduction in the throat deposits. This is favorable for pediatric applications as active substance deposited in the throat generally does not contribute to the therapy, but instead often leads to (systemic) side effects. The use of the add-on device 23 is good as a slight deposit in the throat can only be detected above a flow rate of 20 l/min when the add-on device 23 is used. At all the flow rates, the deposition in the throat with the proposed combination of the inhaler 1 (SMI) with the add-on device 23 is lower than that of the inhaler 1 (SMI) on its own. The DeT can be correlated with the possible therapeutic effect. The proposed combination of the inhaler 1 (SMI) with the add-on device 23 always has a higher DeT than a conventional MDI with the add-on device 23. The loss of DeT by the use of the add-on device 23 compared with the proposed inhaler 1 without the add-on device 23 is detectable but must be estimated as being substantially lower than in a conventional MDI. Therefore, with the proposed solution, a higher efficacy or a higher proportion of active substance reaching the lungs can be assumed.

To complete the disclosure of the present application and with regard to the preferred embodiment of the inhaler 1, the total disclosure of U.S. Pat. No. 5,497,944 and also U.S. Pat. No. 5,964,416 are hereby incorporated by reference.

In contrast to freestanding appliances or the like, the proposed inhaler 1 is preferably designed to be portable and in particular is a mobile hand-held device.

By virtue of its cylindrical shape and handy size of less than 9 to 15 cm long and 2 to 4 cm wide, the inhaler 1 can be carried by the patient at all times. The atomizer sprays a defined volume of the medicament preparation 2 by the application of high pressure through small nozzles, so as to form inhalable aerosols 14.

The proposed inhaler 1 operates purely mechanically, in particular. However, the inhaler 1 may theoretically operate by any other method. In particular, the expression “conveying device” or “pressure generator” must be understood in very general terms. For example, the pressure required for the delivery and atomization may also be produced by propellant gas, a pump or any other suitable method.

The proposed inhaler 1 is designed, in particular, for the brief atomization of the medicament preparation 2, for example for one to two breaths. However, it may also be designed or used for longer or continuous atomization.

Some preferred ingredients, compounds and/or formulations of the medicament preparation 2 are listed below.

The compounds listed below may be used in the device according to the invention on their own or in combination. In the compounds mentioned below, W is a pharmacologically active substance and is selected (for example) from among the betamimetics, anticholinergics, corticosteroids, PDE4-inhibitors, LTD4-antagonists, EGFR-inhibitors, dopamine agonists, H1-antihistamines, PAF-antagonists and PI3-kinase inhibitors. Moreover, double or triple combinations of W may be combined and used in the device according to the invention. Combinations of W might be, for example:

W denotes a betamimetic, combined with an anticholinergic, corticosteroid, PDE4-inhibitor, EGFR-inhibitor or LTD4-antagonist,

W denotes an anticholinergic, combined with a betamimetic, corticosteroid, PDE4-inhibitor, EGFR-inhibitor or LTD4-antagonist,

W denotes a corticosteroid, combined with a PDE4-inhibitor, EGFR-inhibitor or LTD4-antagonist

W denotes a PDE4-inhibitor, combined with an EGFR-inhibitor or LTD4-antagonist

W denotes an EGFR-inhibitor, combined with an LTD4-antagonist.

The compounds used as betamimetics are preferably compounds selected from among albuterol, arformoterol, bambuterol, bitolterol, broxaterol, carbuterol, clenbuterol, fenoterol, formoterol, hexoprenaline, ibuterol, isoetharine, isoprenaline, levosalbutamol, mabuterol, meluadrine, metaproterenol, orciprenaline, pirbuterol, procaterol, reproterol, rimiterol, ritodrine, salmefamol, salmeterol, soterenol, sulphonterol, terbutaline, tiaramide, tolubuterol, zinterol, CHF-1035, HOKU-81, KUL-1248 and

3-(4-{6-[2-hydroxy-2-(4-hydroxy-3-hydroxymethyl-phenyl)-ethylamino]-hexyloxy}-butyl)-benzyl-sulphonamide

5-[2-(5,6-diethyl-indan-2-ylamino)-1-hydroxy-ethyl]-8-hydroxy-1H-quinolin-2-one

4-hydroxy-7-[2-{[2-{[3-(2-phenylethoxy)propyl]sulphonyl}ethyl]-amino}ethyl]-2(3H)-benzothiazolone

1-(2-fluoro-4-hydroxyphenyl)-2-[4-(1-benzimidazolyl)-2-methyl-2-butylamino]ethanol

1-[3-(4-methoxybenzyl-amino)-4-hydroxyphenyl]-2-[4-(1-benzimidazolyl)-2-methyl-2-butylamino]ethanol

1-[2H-5-hydroxy-3-oxo-4H-1,4-benzoxazin-8-yl]-2-[3-(4-N,N-dimethylaminophenyl)-2-methyl-2-propylamino]ethanol

1-[2H-5-hydroxy-3-oxo-4H-1,4-benzoxazin-8-yl]-2-[3-(4-methoxyphenyl)-2-methyl-2-propylamino]ethanol

1-[2H-5-hydroxy-3-oxo-4H-1,4-benzoxazin-8-yl]-2-[3-(4-n-butyloxyphenyl)-2-methyl-2-propylamino]ethanol

1-[2H-5-hydroxy-3-oxo-4H-1,4-benzoxazin-8-yl]-2-{4-[3-(4-methoxyphenyl)-1,2,4-triazol-3-yl]-2-methyl-2-butyl amino }ethanol

5-hydroxy-8-(1-hydroxy-2-isopropylaminobutyl)-2H-1,4-benzoxazin-3-(4H)-one

1-(4-amino-3-chloro-5-trifluoromethylphenyl)-2-tert.-butylamino)ethanol

6-hydroxy-8-{1-hydroxy-2-[2-(4-methoxy-phenyl)-1,1-dimethyl-ethylamino]-ethyl}-4H-benzo[1,4]oxazin-3-one

6-hydroxy-8-{1-hydroxy-2-[2-(ethyl 4-phenoxy-acetate)-1,1-dimethyl-ethylamino]-ethyl}-4H-benzo[1,4]oxazin-3-one

6-hydroxy-8-{1-hydroxy-2-[2-(4-phenoxy-acetic acid)-1,1-dimethyl-ethylamino]-ethyl}-4H-benzo[1,4]oxazin-3-one

8-{2-[1,1-dimethyl-2-(2,4,6-trimethylphenyl)-ethylamino]-1-hydroxy-ethyl}-6-hydroxy-4H-benzo[1,4]oxazin-3-one

6-hydroxy-8-{1-hydroxy-2-[2-(4-hydroxy-phenyl)-1,1-dimethyl-ethylamino]-ethyl}-4H-benzo[1,4]oxazin-3-one

6-hydroxy-8-{1-hydroxy-2-[2-(4-isopropyl-phenyl)-1,1dimethyl-ethylamino]-ethyl}-4H-benzo[1,4]oxazin-3-one

8-{2-[2-(4-ethyl-phenyl)-1,1-dimethyl-ethylamino]-1-hydroxy-ethyl}-6-hydroxy-4H-benzo[1,4]oxazin-3-one

8-{2-[2-(4-ethoxy-phenyl)-1,1-dimethyl-ethylamino]-1-hydroxy-ethyl}-6-hydroxy-4H-benzo[1,4]oxazin-3-one

4-(4-{2-[2-hydroxy-2-(6-hydroxy-3-oxo-3,4-dihydro-2H-benzo[1,4]oxazin-8-yl)-ethylamino]-2-methyl-propyl}-phenoxy)-butyric acid

8-{2-[2-(3,4-difluoro-phenyl)-1,1-dimethyl-ethylamino]-1-hydroxy-ethyl}-6-hydroxy-4H-benzo[1,4]oxazin-3-one

1-(4-ethoxy-carbonylamino-3-cyano-5-fluorophenyl)-2-(tert-butylamino)ethanol

2-hydroxy-5-(1-hydroxy-2-{2-[4-(2-hydroxy-2-phenyl-ethylamino)-phenyl]-ethylamino}-ethyl)-benzaldehyde

N-[2-hydroxy-5-(1-hydroxy-2-{2-[4-(2-hydroxy-2-phenyl-ethylamino)-phenyl]-ethylamino}-ethyl)-phenyl]-formamide

8-hydroxy-5-(1-hydroxy-2-{2-[4-(6-methoxy-biphenyl-3-ylamino)-phenyl]-ethylamino}-ethyl)-1H-quinolin-2-one

8-hydroxy-5-[1-hydroxy-2-(6-phenethylamino-hexylamino)-ethyl]-1H-quinolin-2-one

5-[2-(2-{4-[4-(2-amino-2-methyl-propoxy)-phenylamino]-phenyl}-ethylamino)-1-hydroxy-ethyl]-8-hydroxy-1H-quinolin-2-one

[3-(4-{6-[2-hydroxy-2-(4-hydroxy-3-hydroxymethyl-phenyl)-ethylamino]-hexyloxy}-butyl)-5-methyl-phenyl]-urea

4-(2-{6-[2-(2,6-dichloro-benzyloxy)-ethoxy]-hexylamino}-1-hydroxy-ethyl)-2-hydroxymethyl-phenol

3-(4-{6-[2-hydroxy-2-(4-hydroxy-3-hydroxymethyl-phenyl)-ethylamino]-hexyloxy}-butyl)-benzylsulphonamide

3-(3-{7-[2-hydroxy-2-(4-hydroxy-3-hydroxymethyl-phenyl)-ethylamino]-heptyloxy}-propyl)-benzylsulphonamide

4-(2-{6-[4-(3-cyclopentanesulphonyl-phenyl)-butoxy]-hexylamino}-1-hydroxy-ethyl)-2-hydroxymethyl-phenol

N-Adamantan-2-yl-2-(3-{2-[2-hydroxy-2-(4-hydroxy-3-hydroxymethyl-phenyl)-ethylamino]-propyl}-phenyl)-acetamide

optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The anticholinergics used are preferably compounds selected from among the tiotropium salts, preferably the bromide salt, oxitropium salts, preferably the bromide salt, flutropium salts, preferably the bromide salt, ipratropium salts, preferably the bromide salt, glycopyrronium salts, preferably the bromide salt, trospium salts, preferably the chloride salt, tolterodine. In the above-mentioned salts the cations are the pharmacologically active constituents. As anions the above-mentioned salts may preferably contain the chloride, bromide, iodide, sulphate, phosphate, methanesulphonate, nitrate, maleate, acetate, citrate, fumarate, tartrate, oxalate, succinate, benzoate or p-toluenesulphonate, while chloride, bromide, iodide, sulphate, methanesulphonate or p-toluenesulphonate are preferred as counter-ions. Of all the salts the chlorides, bromides, iodides and methanesulphonates are particularly preferred.

Other preferred anticholinergics are selected from among the salts of formula AC-1

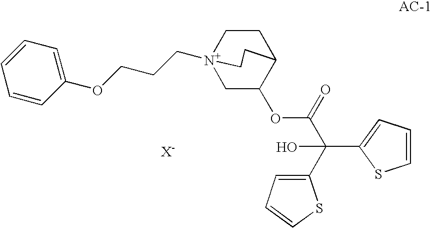

wherein X⁻ denotes an anion with a single negative charge, preferably an anion selected from among the fluoride, chloride, bromide, iodide, sulphate, phosphate, methanesulphonate, nitrate, maleate, acetate, citrate, fumarate, tartrate, oxalate, succinate, benzoate and p-toluenesulphonate, preferably an anion with a single negative charge, particularly preferably an anion selected from among the fluoride, chloride, bromide, methanesulphonate and p-toluenesulphonate, particularly preferably bromide, optionally in the form of the racemates, enantiomers or hydrates thereof. Of particular importance are those pharmaceutical combinations which contain the enantiomers of formula AC-1-en

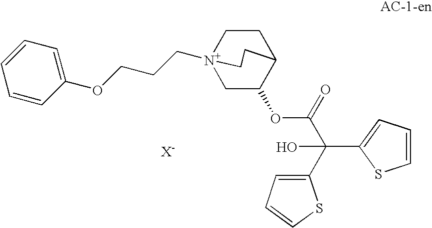

wherein X⁻ may have the above-mentioned meanings. Other preferred anticholinergics are selected from the salts of formula AC-2

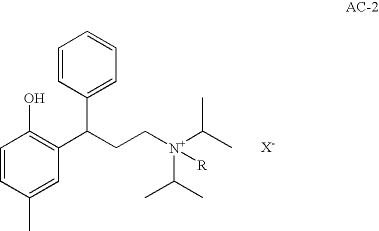

wherein R denotes either methyl or ethyl and wherein X⁻ may have the above-mentioned meanings. In an alternative embodiment the compound of formula AC-2 may also be present in the form of the free base AC-2-base.

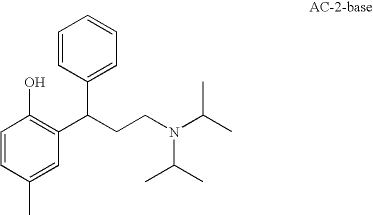

Other specified compounds are:

tropenol 2,2-diphenylpropionate methobromide,

scopine 2,2-diphenylpropionate methobromide,

scopine 2-fluoro-2,2-diphenylacetate methobromide,

tropenol 2-fluoro-2,2-diphenylacetate methobromide;

tropenol 3,3′,4,4′-tetrafluorobenzilate methobromide,

scopine 3,3′,4,4′-tetrafluorobenzilate methobromide,

tropenol 4,4′-difluorobenzilate methobromide,

scopine 4,4′-difluorobenzilate methobromide,

tropenol 3,3′-difluorobenzilate methobromide,

scopine 3,3′-difluorobenzilate methobromide;

tropenol 9-hydroxy-fluorene-9-carboxylate methobromide;

tropenol 9-fluoro-fluorene-9-carboxylate methobromide;

scopine 9-hydroxy-fluorene-9-carboxylate methobromide;

scopine 9-fluoro-fluorene-9-carboxylate methobromide;

tropenol 9-methyl-fluorene-9-carboxylate methobromide;

scopine 9-methyl-fluorene-9-carboxylate methobromide;

cyclopropyltropine benzilate methobromide;

cyclopropyltropine 2,2-diphenylpropionate methobromide;

cyclopropyltropine 9-hydroxy-xanthene-9-carboxylate methobromide;

cyclopropyltropine 9-methyl-fluorene-9-carboxylate methobromide;

cyclopropyltropine 9-methyl-xanthene-9-carboxylate methobromide;

cyclopropyltropine 9-hydroxy-fluorene-9-carboxylate methobromide;

cyclopropyltropine methyl 4,4′-difluorobenzilate methobromide.

tropenol 9-hydroxy-xanthene-9-carboxylate methobromide;

scopine 9-hydroxy-xanthene-9-carboxylate methobromide;

tropenol 9-methyl-xanthene-9-carboxylate methobromide;

scopine 9-methyl-xanthene-9-carboxylate methobromide;

tropenol 9-ethyl-xanthene-9-carboxylate methobromide;

tropenol 9-difluoromethyl-xanthene-9-carboxylate methobromide;

scopine 9-hydroxymethyl-xanthene-9-carboxylate methobromide,

The above-mentioned compounds may also be used as salts within the scope of the present invention, wherein instead of the methobromide the metho-X salts are used, wherein X may have the meanings given hereinbefore for X⁻.

As corticosteroids it is preferable to use compounds selected from among beclomethasone, betamethasone, budesonide, butixocort, ciclesonide, deflazacort, dexamethasone, etiprednol, flunisolide, fluticasone, loteprednol, mometasone, prednisolone, prednisone, rofleponide, triamcinolone, RPR-106541, NS-126, ST-26 and

(S)-fluoromethyl 6,9-difluoro-17-[(2-furanylcarbonyl)oxy]-11-hydroxy-16-methyl-3-oxo-androsta-1,4-diene-17-carbothionate

(S)-(2-oxo-tetrahydro-furan-3S-yl)6,9-difluoro-11-hydroxy-16-methyl-3-oxo-17-propionyloxy-androsta-1,4-diene-17-carbothionate,

Cyanomethyl 6α,9α-difluoro-11β-hydroxy-16α-methyl-3-oxo-17α-(2,2,3,3-tertamethylcyclopropylcarbonyl)oxy-androsta-1,4-diene-17β-carboxylate optionally in the form of the racemates, enantiomers or diastereomers thereof and optionally in the form of the salts and derivatives thereof, the solvates and/or hydrates thereof. Any reference to steroids includes a reference to any salts or derivatives, hydrates or solvates thereof which may exist. Examples of possible salts and derivatives of the steroids may be: alkali metal salts, such as for example sodium or potassium salts, sulphobenzoates, phosphates, isonicotinates, acetates, dichloroacetates, propionates, dihydrogen phosphates, palmitates, pivalates or furoates.

PDE4-inhibitors which may be used are preferably compounds selected from among enprofyllin, theophyllin, roflumilast, ariflo (cilomilast), tofimilast, pumafentrin, lirimilast, arofyllin, atizoram, D-4418, Bay-198004, BY343, CP-325.366, D-4396 (Sch-351591), AWD-12-281 (GW-842470), NCS-613, CDP-840, D-4418, PD-168787, T-440, T-2585, V-11294A, C1-1018, CDC-801, CDC-3052, D-22888, YM-58997, Z-15370 and

N-(3,5-dichloro-1-oxo-pyridin-4-yl)-4-difluoromethoxy-3-cyclopropylmethoxybenzamide

(−)p-[(4aR*,10bS*)-9-ethoxy-1,2,3,4,4a,10b-hexahydro-8-methoxy-2-methylbenzo[s] [1,6]naphthyridin-6-yl]-N,N-diisopropylbenzamide

(R)-(+)-1-(4-bromobenzyl)-4-[(3-cyclopentyloxy)-4-methoxyphenyl]-2-pyrrolidone

3-(cyclopentyloxy-4-methoxyphenyl)-1-(4-N′-[N-2-cyano-S-methyl-isothioureido]benzyl)-2-pyrrolidone

cis[4-cyano-4-(3-cyclopentyloxy-4-methoxyphenyl)cyclohexane-1-carboxylic acid]

2-carbomethoxy-4-cyano-4-(3-cyclopropylmethoxy-4-difluoromethoxy-phenyl)cyclohexan-1-one

cis[4-cyano-4-(3-cyclopropylmethoxy-4-difluoromethoxyphenyl)cyclohexan-1-ol]

(R)-(+)-ethyl[4-(3-cyclopentyloxy-4-methoxyphenyl)pyrrolidin-2-ylidene]acetate

(S)-(−)-ethyl[4-(3-cyclopentyloxy-4-methoxyphenyl)pyrrolidin-2-ylidene]acetate

9-cyclopentyl-5,6-dihydro-7-ethyl-3-(2-thienyl)-9H-pyrazolo[3,4-c]-1,2,4-triazolo[4,3-a]pyridine

9-cyclopentyl-5,6-dihydro-7-ethyl-3-(tert-butyl)-9H-pyrazolo[3,4-c]-1,2,4-triazolo[4,3-a]pyridine

optionally in the form of the racemates, enantiomers or diastereomers thereof and optionally in the form of the pharmacologically acceptable acid addition salts thereof, the solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The LTD4-antagonists used are preferably compounds selected from among montelukast, pranlukast, zafirlukast, MCC-847 (ZD-3523), MN-001, MEN-91507 (LM-1507), VUF-5078, VUF-K-8707, L-733321 and

1-(((R)-(3-(2-(6,7-difluoro-2-quinolinyl)ethenyl)phenyl)-3-(2-(2-hydroxy-2-propyl)phenyl)thio)methylcyclopropane-acetic acid,

1-(((1(R)-3(3-(2-(2,3-dichlorothieno[3,2-b]pyridin-5-yI)-(E)-ethenyl)phenyl)-3-(2-(1-hydroxy-1-methylethyl)phenyl)propyl)thio)methyl)cyclopropaneacetic acid

[2-[[2-(4-tert-butyl-2-thiazolyl)-5-benzofuranyl]oxymethyl]phenyl]acetic acid

optionally in the form of the racemates, enantiomers or diastereomers thereof and optionally in the form of the pharmacologically acceptable acid addition salts, solvates and/or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate. By salts or derivatives which the LTD4-antagonists may optionally be capable of forming are meant, for example: alkali metal salts, such as for example sodium or potassium salts, alkaline earth metal salts, sulphobenzoates, phosphates, isonicotinates, acetates, propionates, dihydrogen phosphates, palmitates, pivalates or furoates.

EGFR-inhibitors which may be used are preferably compounds selected from among cetuximab, trastuzumab, ABX-EGF, Mab ICR-62 and

4-[(3-chloro-4-fluorophenyl)amino]-6-{[4-(morpholin-4-yl)-1-oxo-2-buten-1-yl]amino}-7-cyclopropylmethoxy-quinazoline

4-[(3-chloro-4-fluorophenyl)amino]-6-{[4-(N,N-diethylamino)-1-oxo-2-buten-1-yl]-amino}-7-cyclopropylmethoxy-quinazoline

4-[(3-chloro-4-fluorophenyl)amino]-6-{[4-(N,N-dimethylamino)-1-oxo-2-buten-1-yl]amino}-7-cyclopropylmethoxy-quinazoline

4-[(R)-(1-phenyl-ethyl)amino]-6-{[4-(morpholin-4-yl)-1-oxo-2-buten-1-yl]amino}-7-cyclopentyloxy-quinazoline

4-[(3-chloro-4-fluoro-phenyl)amino]-6-{[4-((R)-6-methyl-2-oxo-morpholin-4-yl)-1-oxo-2-buten-1-yl]amino}-7-cyclopropylmethoxy-quinazoline

4-[(3-chloro-4-fluoro-phenyl)amino]-6-{[4-((R)-6-methyl-2-oxo-morpholin-4-yl)-1-oxo-2-buten-1-yl]amino}-7-[(S)-(tetrahydrofuran-3-yl)oxy]-quinazoline

4-[(3-chloro-4-fluoro-phenyl)amino]-6-{[4-((R)-2-methoxymethyl-6-oxo-morpholin-4-yl)-1-oxo-2-buten-1-yl]amino}-7-cyclopropylmethoxy-quinazoline

4-[(3-chloro-4-fluoro-phenyl)amino]-6-[2-((S)-6-methyl-2-oxo-morpholin-4-yl)-ethoxy]-7-methoxy-quinazoline

4-[(3-chloro-4-fluorophenyl)amino]-6-({4-[N-(2-methoxy-ethyl)-N-methyl-amino]-1-oxo-2-buten-1-yl}amino)-7-cyclopropylmethoxy-quinazoline

4-[(3-chloro-4-fluorophenyl)amino]-6-{[4-(N,N-dimethylamino)-1-oxo-2-buten-1-yl]amino}-7-cyclopentyloxy-quinazoline

4-[(R)-(1-phenyl-ethyl)amino]-6-{[4-(N,N-to-(2-methoxy-ethyl)-amino)-1-oxo-2-buten-1-yl]amino}-7-cyclopropylmethoxy-quinazoline

4-[(R)-(1-phenyl-ethyl)amino]-6-({4-[N-(2-methoxy-ethyl)-N-ethyl-amino]-1-oxo-2-buten-1-yl}amino)-7-cyclopropylmethoxy-quinazoline

4-[(R)-(1-phenyl-ethyl)amino]-6-({4-[N-(2-methoxy-ethyl)-N-methyl-amino]-1-oxo-2-buten-1-yl}amino)-7-cyclopropylmethoxy-quinazoline

4-[(R)-(1-phenyl-ethyl)amino]-6-({4-[N-(tetrahydropyran-4-yl)-N-methyl-amino]-1-oxo-2-buten-1-yl}amino)-7-cyclopropylmethoxy-quinazoline

4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline

4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline

4-[(3-chloro-4-fluorophenyl)amino]-6-({4-[N-(2-methoxy-ethyl)-N-methyl-amino]-1-oxo-2-buten-1-yl}amino)-7-cyclopentyloxy-quinazoline

4-[(3-chloro-4-fluorophenyl)amino]-6-{[4-(N-cyclopropyl-N-methyl-amino)-1-oxo-2-buten-1-yl]amino}-7-cyclopentyloxy-quinazoline

4-[(3-chloro-4-fluorophenyl)amino]-6-{[4-(N,N-dimethylamino)-1-oxo-2-buten-1-yl]amino}-7-[(R)-(tetrahydrofuran-2-yl)methoxy]-quinazoline

4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline

4-[(3-ethynyl-phenyl)amino]-6,7-to-(2-methoxy-ethoxy)-quinazoline

4-[(3-chloro-4-fluorophenyl)amino]-7-[3-(morpholin-4-yl)-propyloxy]-6-[(vinyl-carbonyl)amino]-quinazoline

4-[(R)-(1-phenyl-ethyl)amino]-6-(4-hydroxy-phenyl)-7H-pyrrolo[2,3-d]pyrimidine

3-cyano-4-[(3-chloro-4-fluorophenyl)amino]-6-{[4-(N,N-dimethylamino)-1-oxo-2-buten-1-yl]amino}-7-ethoxy-quinoline

4-{[3-chloro-4-(3-fluoro-benzyloxy)-phenyl]amino}-6-(5-{[(2-methanesulphonyl-ethyl)amino]methyl}-furan-2-yl)quinazoline

4-[(R)-(1-phenyl-ethyl)amino]-6-{[4-((R)-6-methyl-2-oxo-morpholin-4-yl)-1-oxo-2-buten-1-yl]amino}-7-methoxy-quinazoline

4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline

4-[(3-chloro-4-fluorophenyl)amino]-6-({4-[N,N-to-(2-methoxy-ethyl)-amino]-1-oxo-2-buten-1-yl}amino)-7-[(tetrahydrofuran-2-yl)methoxy]-quinazoline

4-[(3-ethynyl-phenyl)amino]-6-{[4-(5,5-dimethyl-2-oxo-morpholin-4-yl)-1-oxo-2-buten-1-yl]amino}-quinazoline

4-[(3-chloro-4-fluoro-phenyl)amino]-6-[2-(2,2-dimethyl-6-oxo-morpholin-4-yl)-ethoxy]-7-methoxy-quinazoline

4-[(3-chloro-4-fluoro-phenyl)amino]-6-[2-(2,2-dimethyl-6-oxo-morpholin-4-yl)-ethoxy]-7-[(R)-(tetrahydrofuran-2-yl)methoxy]-quinazoline

4-[(3-chloro-4-fluoro-phenyl)amino]-7-[2-(2,2-dimethyl-6-oxo-morpholin-4-yl)-ethoxy]-6-[(S)-(tetrahydrofuran-2-yl)methoxy]-quinazoline

4-[(3-chloro-4-fluoro-phenyl)amino]-6-{2-[4-(2-oxo-morpholin-4-yl)-piperidin-1-yl]-ethoxy}-7-methoxy-quinazoline

4-[(3-chloro-4-fluoro-phenyl)amino]-6-[1-(tert.-butyloxycarbonyl)-piperidin-4-yloxy]-7-methoxy-quinazoline

4-[(3-chloro-4-fluoro-phenyl)amino]-6-(trans-4-amino-cyclohexan-1-yloxy)-7-methoxy-quinazoline

4-[(3-chloro-4-fluoro-phenyl)amino]-6-(trans-4-methanesulphonylamino-cyclohexan-1-yloxy)-7-methoxy-quinazoline

4-[(3-chloro-4-fluoro-phenyl)amino]-6-(tetrahydropyran-3-yloxy)-7-methoxy-quinazoline

4-[(3-chloro-4-fluoro-phenyl)amino]-6-(1-methyl-piperidin-4-yloxy)-7-methoxy-quinazoline

4-[(3-chloro-4-fluoro-phenyl)amino]-6-{1-[(morpholin-4-yl)carbonyl]-piperidin-4-yl-oxy}-7-methoxy-quinazoline

4-[(3-chloro-4-fluoro-phenyl)amino]-6-{1-[(methoxymethyl)carbonyl]-piperidin-4-yl-oxy}-7-methoxy-quinazoline

4-[(3-chloro-4-fluoro-phenyl)amino]-6-(piperidin-3-yloxy)-7-methoxy-quinazoline

4-[(3-chloro-4-fluoro-phenyl)amino]-6-[1-(2-acetylamino-ethyl)-piperidin-4-yloxy]-7-methoxy-quinazoline

4-[(3-chloro-4-fluoro-phenyl)amino]-6-(tetrahydropyran-4-yloxy)-7-ethoxy-quinazoline

4-[(3-chloro-4-fluoro-phenyl)amino]-6-((S)-tetrahydrofuran-3-yloxy)-7-hydroxy-quinazoline

4-[(3-chloro-4-fluoro-phenyl)amino]-6-(tetrahydropyran-4-yloxy)-7-(2-methoxy-ethoxy)-quinazoline

4-[(3-chloro-4-fluoro-phenyl)amino]-6-{trans-4-[(dimethylamino)sulphonylamino]-cyclohexan-1-yloxy}-7-methoxy-quinazoline

4-[(3-chloro-4-fluoro-phenyl)amino]-6-{trans-4-[(morpholin-4-yl)carbonylamino]-cyclohexan-1-yloxy}-7-methoxy-quinazoline

4-[(3-chloro-4-fluoro-phenyl)amino]-6-{trans-4-[(morpholin-4-yl)sulphonylamino]-cyclohexan-1-yloxy}-7-methoxy-quinazoline

4-[(3-chloro-4-fluoro-phenyl)amino]-6-(tetrahydropyran-4-yloxy)-7-(2-acetylamino-ethoxy)-quinazoline

4-[(3-chloro-4-fluoro-phenyl)amino]-6-(tetrahydropyran-4-yloxy)-7-(2-methanesulphonylamino-ethoxy)-quinazoline

4-[(3-chloro-4-fluoro-phenyl)amino]-6-{1-[(piperidin-1-yl)carbonyl]-piperidin-4-yloxy}-7-methoxy-quinazoline

4-[(3-chloro-4-fluoro-phenyl)amino]-6-(1-aminocarbonylmethyl-piperidin-4-yloxy)-7-methoxy-quinazoline

4-[(3-chloro-4-fluoro-phenyl)amino]-6-(cis-4-{N-[(tetrahydropyran-4-yl)carbonyl]-N-methyl-amino}-cyclohexan-1-yloxy)-7-methoxy-quinazoline

4-[(3-chloro-4-fluoro-phenyl)amino]-6-(cis-4-{N-[(morpholin-4-yl)carbonyl]-N-methyl-amino}-cyclohexan-1-yloxy)-7-methoxy-quinazoline

4-[(3-chloro-4-fluoro-phenyl)amino]-6-(cis-4-{N-[(morpholin-4-yl)sulphonyl]-N-methyl-amino}-cyclohexan-1-yloxy)-7-methoxy-quinazoline

4-[(3-chloro-4-fluoro-phenyl)amino]-6-(trans-4-ethanesulphonylamino-cyclohexan-1-yloxy)-7-methoxy-quinazoline

4-[(3-chloro-4-fluoro-phenyl)amino]-6-(1-methanesulphonyl-piperidin-4-yloxy)-7-ethoxy-quinazoline

4-[(3-chloro-4-fluoro-phenyl)amino]-6-(1-methanesulphonyl-piperidin-4-yloxy)-7-(2-methoxy-ethoxy)-quinazoline

4-[(3-chloro-4-fluoro-phenyl)amino]-6-[1-(2-methoxy-acetyl)-piperidin-4-yloxy]-7-(2-methoxy-ethoxy)-quinazoline

4-[(3-chloro-4-fluoro-phenyl)amino]-6-(cis-4-acetylamino-cyclohexan-1-yloxy)-7-methoxy-quinazoline

4-[(3-ethynyl-phenyl)amino]-6-[1-(tert.-butyloxycarbonyl)-piperidin-4-yloxy]-7-methoxy-quinazoline

4-[(3-ethynyl-phenyl)amino]-6-(tetrahydropyran-4-yloxy]-7-methoxy-quinazoline

4-[(3-chloro-4-fluoro-phenyl)amino]-6-(cis-4-{N-[(piperidin-1-yl)carbonyl]-N-methyl-amino}-cyclohexan-1-yloxy)-7-methoxy-quinazoline

4-[(3-chloro-4-fluoro-phenyl)amino]-6-(cis-4-{N-[(4-methyl-piperazin-1-yl)carbonyl]-N-methyl-amino}-cyclohexan-1-yloxy)-7-methoxy-quinazoline

4-[(3-chloro-4-fluoro-phenyl)amino]-6-{cis-4-[(morpholin-4-yl)carbonylamino]-cyclohexan-1-yloxy}-7-methoxy-quinazoline

4-[(3-chloro-4-fluoro-phenyl)amino]-6-{1-[2-(2-oxopyrrolidin-1-yl)ethyl]-piperidin-4-yloxy}-7-methoxy-quinazoline

4-[(3-chloro-4-fluoro-phenyl)amino]-6-{1-[(morpholin-4-yl)carbonyl]-piperidin-4-yloxy}-7-(2-methoxy-ethoxy)-quinazoline

4-[(3-ethynyl-phenyl)amino]-6-(1-acetyl-piperidin-4-yloxy)-7-methoxy-quinazoline

4-[(3-ethynyl-phenyl)amino]-6-(1-methyl-piperidin-4-yloxy)-7-methoxy-quinazoline

4-[(3-ethynyl-phenyl)amino]-6-(1-methanesulphonyl-piperidin-4-yloxy)-7-methoxy-quinazoline

4-[(3-chloro-4-fluoro-phenyl)amino]-6-(1-methyl-piperidin-4-yloxy)-7(2-methoxy-ethoxy)-quinazoline

4-[(3-chloro-4-fluoro-phenyl)amino]-6-(1-isopropyloxycarbonyl-piperidin-4-yloxy)-7-methoxy-quinazoline

4-[(3-chloro-4-fluoro-phenyl)amino]-6-(cis-4-methylamino-cyclohexan-1-yloxy)-7-methoxy-quinazoline

4-[(3-chloro-4-fluoro-phenyl)amino]-6-{cis-4-[N-(2-methoxy-acetyl)-N-methyl-amino]-cyclohexan-1-yloxy}-7-methoxy-quinazoline

4-[(3-ethynyl-phenyl)amino]-6-(piperidin-4-yloxy)-7-methoxy-quinazoline

4-[(3-ethynyl-phenyl)amino]-6-[1-(2-methoxy-acetyl)-piperidin-4-yloxy]-7-methoxy-quinazoline

4-[(3-ethynyl-phenyl)amino]-6-{1-[(morpholin-4-yl)carbonyl]-piperidin-4-yloxy}-7-methoxy-quinazoline

4-[(3-chloro-4-fluoro-phenyl)amino]-6-{1-[(cis-2,6-dimethyl-morpholin-4-yl)carbonyl]-piperidin-4-yloxy}-7-methoxy-quinazoline

4-[(3-chloro-4-fluoro-phenyl)amino]-6-{1-[(2-methyl-morpholin-4-yl)carbonyl]-piperidin-4-yloxy}-7-methoxy-quinazoline

4-[(3-chloro-4-fluoro-phenyl)amino]-6-{1-[(S,S)-(2-oxa-5-aza-bicyclo[2,2,1]hept-5-yl)carbonyl]-piperidin-4-yloxy}-7-methoxy-quinazoline

4-[(3-chloro-4-fluoro-phenyl)amino]-6-{1-[(N-methyl-N-2-methoxyethyl-amino)carbonyl]-piperidin-4-yloxy}-7-methoxy-quinazoline

4-[(3-chloro-4-fluoro-phenyl)amino]-6-(1-ethyl-piperidin-4-yloxy)-7-methoxy-quinazoline

4-[(3-chloro-4-fluoro-phenyl)amino]-6-{1-[(2-methoxyethyl)carbonyl]-piperidin-4-yloxy}-7-methoxy-quinazoline

4-[(3-chloro-4-fluoro-phenyl)amino]-6-{1-[(3-methoxypropyl-amino)-carbonyl]-piperidin-4-yloxy}-7-methoxy-quinazoline

4-[(3-chloro-4-fluoro-phenyl)amino]-6-[cis-4-(N-methanesulphonyl-N-methyl-amino)-cyclohexan-1-yloxy]-7-methoxy-quinazoline

4-[(3-chloro-4-fluoro-phenyl)amino]-6-[cis-4-(N-acetyl-N-methyl-amino)-cyclohexan-1-yloxy]-7-methoxy-quinazoline

4-[(3-chloro-4-fluoro-phenyl)amino]-6-(trans-4-methylamino-cyclohexan-1-yloxy)-7-methoxy-quinazoline

4-[(3-chloro-4-fluoro-phenyl)amino]-6-[trans-4-(N-methanesulphonyl-N-methyl-amino)-cyclohexan-1-yloxy]-7-methoxy-quinazoline

4-[(3-chloro-4-fluoro-phenyl)amino]-6-(trans-4-dimethylamino-cyclohexan-1-yloxy)-7-methoxy-quinazoline

4-[(3-chloro-4-fluoro-phenyl)amino]-6-(trans-4-{N-[(morpholin-4-yl)carbonyl]-N-methyl-amino}-cyclohexan-1-yloxy)-7-methoxy-quinazoline

4-[(3-chloro-4-fluoro-phenyl)amino]-6-[2-(2,2-dimethyl-6-oxo-morpholin-4-yl)-ethoxy]-7-[(S)-(tetrahydrofuran-2-yl)methoxy]-quinazoline

4-[(3-chloro-4-fluoro-phenyl)amino]-6-(1-methanesulphonyl-piperidin-4-yloxy)-7-methoxy-quinazoline

4-[(3-chloro-4-fluoro-phenyl)amino]-6-(1-cyano-piperidin-4-yloxy)-7-methoxy-quinazoline

optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

The dopamine agonists used are preferably compounds selected from among bromocriptin, cabergoline, alpha-dihydroergocryptine, lisuride, pergolide, pramipexol, roxindol, ropinirol, talipexol, tergurid and viozan, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydrooxalate, hydrosuccinate, hydrobenzoate and hydro-p-toluenesulphonate.

H1-Antihistamines which may be used are preferably compounds selected from among epinastine, cetirizine, azelastine, fexofenadine, levocabastine, loratadine, mizolastine, ketotifen, emedastine, dimetindene, clemastine, bamipine, cexchlorpheniramine, pheniramine, doxylamine, chlorophenoxamine, dimenhydrinate, diphenhydramine, promethazine, ebastine, desloratidine and meclozine, optionally in the form of the racemates, enantiomers, diastereomers thereof and optionally in the form of the pharmacologically acceptable acid addition salts, solvates or hydrates thereof. According to the invention the acid addition salts of the betamimetics are preferably selected from among the hydrochloride, hydrobromide, hydriodide, hydrosulphate, hydrophosphate, hydromethanesulphonate, hydronitrate, hydromaleate, hydroacetate, hydrocitrate, hydrofumarate, hydrotartrate, hydroxalate, hydrosuccinate, hydrobenzoate and hydro-p-toluenesulphonate.

In addition, inhalable macromolecules as disclosed in EP 1 003 478 A1 or CA 2297174 A1 may also be used.

In addition, the compound may be selected from among the ergot alkaloid derivatives, the triptans, the CGRP-inhibitors, the phosphodiesterase-V inhibitors, optionally in the form of the racemates, enantiomers or diastereomers thereof, optionally in the form of the pharmacologically acceptable acid addition salts, the solvates and/or hydrates thereof.

Examples of ergot alkaloid derivatives are dihydroergotamine and ergotamine.

What is claimed is:

1. Portable inhaler for the propellant-free atomization of a medicament preparation, comprising:
a pressure generator and
a delivery nozzle for delivering the atomized medicament preparation as an aerosol, and
an add-on device with a chamber for intermediate storage of the aerosol,
wherein the chamber is mountable downstream of the delivery nozzle.

2. Inhaler according to claim 1, further comprising a mouthpiece into which the atomized medicament preparation deliverable from the nozzle.

3. Inhaler according to claim 2, wherein the add-on device is mountable onto the mouthpiece.

4. Inhaler according to claim 2, wherein the add-on device is removably mountable on mouthpiece.

5. Inhaler according to claim 2, wherein the add-on device comprises a valve for at least one of: preventing air from flowing back into the chamber or mouthpiece; and sucking in of the aerosol.

6. Inhaler according to claim 5, wherein the valve is located at the delivery end of the add-on device.

7. Inhaler according to claim 1, wherein the chamber of the add-on device is at least one of substantially cylindrical, elongate and conical in construction.

8. Inhaler according to claim 1, wherein the chamber has a volume of more than 0.1 l

9. Inhaler according to claim 1, wherein the chamber has a volume of about 0.2 to 0.6 l.

10. Inhaler according to claim 2, wherein the mouthpiece has at least one supply air opening which remains open when the add-on device is attached.

11. Inhaler according to claim 1, wherein the add-on device is adapted to be equipped with at least one of an add-on mouthpiece, tube and face mask.

12. Inhaler according claim 1, wherein the inhaler is constructed to expel the aerosol at a speed of less than 2 m/s at a distance of 10 cm from the delivery nozzle.

13. Inhaler according to claim 1, wherein the inhaler is constructed to deliver and atomize 10 to 50 μl of the medicament preparation over a period of at least 1 s on each actuation or dose.

14. Inhaler according to claim 1, wherein the inhaler is constructed to atomize defined amounts of the medicament preparation at a pressure of 10 to 60 MPa.

15. Inhaler according to claim 1, wherein the atomization is by the pressure generator produced by spring force.

16. Inhaler according to claim 1, wherein the pressure generator is constructed as a pump.

17. Inhaler according to claim 1, wherein the pressure generator is mechanically operable.

18. Inhaler according to claim 1, wherein the add-on device comprises at least one valve for allowing exhaled air to be blown out.

19. Inhaler according to claim 1, wherein the inhaler is a soft mist inhaler.

20. Portable inhaler for propellant-free atomization of a medicament preparation, comprising a pressure generator pump, a delivery nozzle adapted to deliver the medicament preparation as an aerosol at a speed of less than 2 m/s at a distance of 10 cm from the delivery nozzle, and an add-on device with a chamber for intermediate storage of the atomized medicament preparation, the chamber being connected to the inhaler downstream of the delivery nozzle.
